(19) United States
(12) Patent Application Publication
(10) Pub. No.: US 2017/0053362 A1
(43) Pub. Date: Feb. 23, 2017

(54) Methods and Systems for a Gastronomic Recommendation Engine

(71) Applicant: Tastegraphy, Inc., Austin, TX (US)

(72) Inventors: Federico Galarraga, Austin, TX (US); Mokshika Sharma, Austin, TX (US)

(21) Appl. No.: 14/829,030

(22) Filed: Aug. 18, 2015

(51) Int. Cl. G06Q 50/12; G06Q 30/06; G01N 33/00

(52) U.S. Cl. CPC G06Q 50/12; G01N 33/0001; G06Q 30/0631

(57) ABSTRACT

Food recommendations are produced from a taste match between a user categorized by the user's taste preferences and a Taste Item categorized by its taste and offered by a food provider.

### CROSS-REFERENCE TO RELATED APPLICATIONS

The present application is related to, a continuation-in-part of, claims priority from and the benefit of U.S. patent application Ser. No. 14/829,030, filed Aug. 18, 2015, having the same title and the same inventors, the disclosure of which is incorporated herein by reference in its entirety.

### TECHNICAL FIELD

This disclosure relates generally to taste and food matching methods and systems, and more particularly to methods and systems for recommending food that matches a user's taste profile.

### BACKGROUND

Finding what to eat is often a challenge. A tool, system, or method that provides accurate, user-specific, restaurant dish recommendations would be very useful.

### SUMMARY

The present disclosure describes a method for a user to obtain accurate, user-specific, restaurant dish recommendations. Specific exemplary embodiments of the method feature a Recommendation Engine that may be implemented on a tool, such as a software app, that constantly learns a user's taste preferences based on the ratings given to each dish by the user, and based on viewing and searching habits inside of the app.

The present disclosure describes methods and processes to produce a taste recommendation between a user and a Taste Item offered by a food provider categorized by the user's taste preferences. The user's taste preferences include the user's dietary requirements.

The present disclosure also describes methods and processes to initialize, populate, gather, evolve, learn, and understand a user's taste preferences.

The present disclosure describes methods and processes to identify the good and bad combinations of Taste Tags for a specific user.

The present disclosure describes methods and processes to evolve a user's taste preferences based on their actions such as viewing, searching, liking, rating, and so forth.

The present disclosure describes methods and processes to create and populate a Taste Item database through the curated input of a Taste Expert.

The present disclosure describes methods and processes to produce a Recommendation Engine result between a user and a consumable or edible Item categorized by the Item's taste. The result is displayed to the user in one or more suitable ways, such as numerical, graphical, auditory, and the like.

The present disclosure describes methods and processes to render on a screen the Recommendation Engine result between a user and a consumable or edible Item categorized by the Item's taste.

The present disclosure describes methods and processes to categorize an edible or consumable Item based on a predetermined and evolving Taste Network.

The present disclosure describes method and process to create, curate, maintain, and expand a Taste Network by a Taste Expert.

### BRIEF DESCRIPTION OF THE DRAWINGS

For a more complete understanding of the present disclosure, and the advantages thereof, reference is now made to the following descriptions taken in conjunction with the accompanying drawings, in which:

FIG. 1 is a process flowchart for an exemplary embodiment of a Recommendation Engine of the present disclosure.

FIG. 2 is a process flow chart for an exemplary embodiment of a rating system of a Recommendation Engine of the present disclosure.

FIG. 3 is a process flow chart for an exemplary embodiment of a matching tool of a Recommendation Engine of the present disclosure.

FIG. 4 is a process flow chart for an exemplary embodiment of a taste database of a Recommendation Engine of the present disclosure.

FIG. 5 is a diagrammatic illustration of exemplary embodiments of Taste Item databases of a system of the present disclosure.

FIG. 6 is a diagrammatic illustration of exemplary embodiments of user databases of a system of the present disclosure.

FIG. 7 is a diagrammatic representation of a machine in the form of a computer system within which a set of instructions, when executed, may cause the machine to perform any one or more of the methodologies discussed herein.

### DETAILED DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

In the following discussion and in the claims, the terms “including” and “comprising” are used in an open-ended fashion, and thus should be interpreted to mean “including, but not limited to . . . .” Also, the term “link” or “points to,” and forms thereof, are intended to mean either an indirect or direct connection. Thus, if a first component links to a second component, that connection may be through a direct connection or through an indirect connection via other components and connections.

Certain terms are used throughout the following description and claims to refer to particular system components and method steps. As one skilled in the art will appreciate, different products may refer to a component by different names. This document does not intend to distinguish between components that differ in name but not function. The following defined terms and phrases are used in the description of the Recommendation Engine methods and processes:

Taste Expert: A human or non-human entity that follows a defined process to categorize a Taste Item using Tags in the Taste Network.

Taste Network: User curated hierarchical graph that relates the Taste Tags to each other and between themselves. The Taste Network is curated by a Taste Expert and can be enhanced through: user actions, forced temporary or permanent relations, automated learning, Internet crawling, Application Program Interface (“API”) callers, and so forth.

Taste Tags (“Tags”): Set of words that fall into one of the 17 unique Tag types. They are used to describe every possible component of taste. They are curated by a Taste Expert and can be enhanced through: user actions, admin actions, Taste Experts, Internet crawling, and API callers.

Unique Tag Types:

Cuisine type

Ingredients

Textures

Aromas

Cooking methods

Temperature

Dish type

Taste intensity profile

Mood

Size

Dietary description

Time

Density

Occasion

Seasonality

Region

Weather

Taste Item or Item: Any edible or consumable thing offered by a food provider, including, but not limited to, food dishes, menu entries, ingredients, spices, and sauces, as well as drinks, cocktails, refreshments and desserts.

All data in the present Recommendation Engine can be tweaked, enhanced, or modified by a Taste Expert. Data is stored in databases such as the following:

User Databases: Modified by admin, user, API caller. Each user database is created with initial content provided by the API caller, user, admin, or through a quiz, survey, or the user's Internet profiles such as Instagram, Facebook, and the like. All data can be updated and re-fetched as long as the user has an entry in the database. User databases consist of a preference database, which holds the user's preference information towards each Tag in the Taste network. Each preference entry consists of pairings databases, which hold the user's preference information for the combination between that preference and another preference. The user databases are constantly evolving through the rating system to generate a user taste profile expressed, for example, of Taste Tags.

Taste Item Database: Modified by admin, user, and/or API caller. It is created with initial content provided by the API caller, user, admin, through a quiz, survey, or from the owner's Internet profiles. It is comprised of Taste Tags that are part of the Taste Network.

All databases record the following factors: date, time, user data (gender, age, ethnicity, and so forth), location of API/request caller, current weather at the location of the caller, current local events at location of caller, and taste information requested.

Analytics are recorded when the following actions are performed: viewing, searching, rating a Taste Item, and app specific interactions.

The engine works in direct conjunction with a specific database schema, multiple engines, and setup that utilizes a food-specific set of Tags that are connected in a network based on their relationships.

The Recommendation Engine is comprised of 3 subsystems that are required for its complete functionality:

Rating System—When the user expresses interest in an Item, the engine records all the possible combinations of all the Tags owned by that dish in a preference database specific for that user. This also records the frequency of these combinations alongside with the rating provided, which allows them to be recorded as good pairings and bad pairings.

The user has a preference database that records the frequency and average rating for each Tag the user has expressed any interested in, as well as a pointer to 2 separate databases good pairing and bad pairing, to record the combinational relationships between Tags for that user.

Matching Algorithm—When a recommendation is calculated, it evaluates an Item's set of Tags against the preference database to see how many good and bad pairings it includes. It then evaluates the frequency of that preference and pairing for that user and changes its weight based on the accumulated average rating for that specific Tag. The final score is calculated as a difference of the total good pairing weighted frequency and the total bad pairing weighted frequency. A final percentage match is also calculated to provide a confidence factor for the final score. This percentage takes into account an initial set of Tag preferences provided by the user, as well as dietary requirements.

Taste Network—All taste data is stored in a curated relational network of Taste Tags that is comprised of words that fall into the set of 17 unique Tag types.

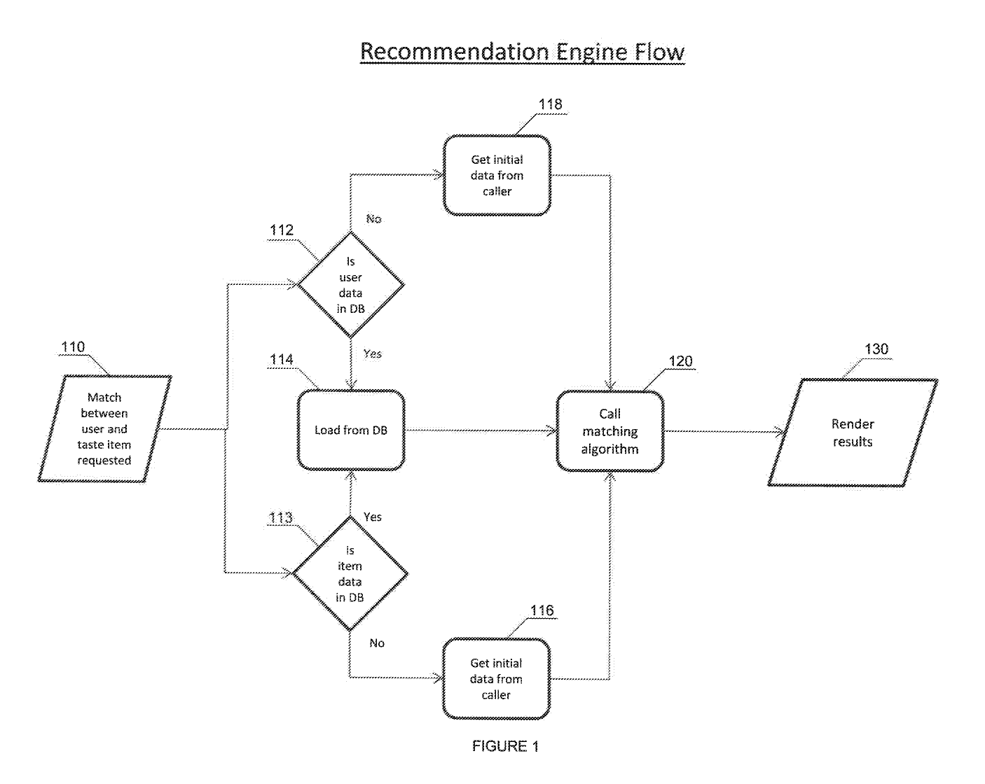

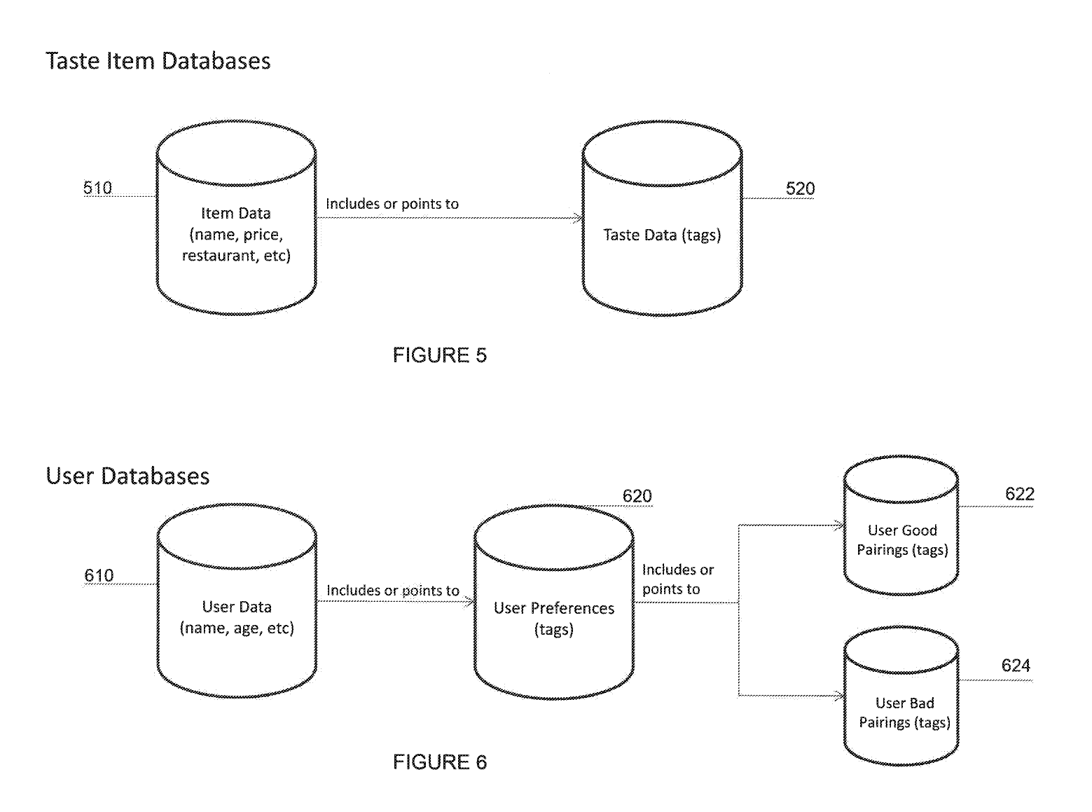

Turning now to the drawings and referring to FIG. 1, FIG. 1 is a process flowchart for an exemplary embodiment of a Recommendation Engine of the present disclosure. A match between a user and a Taste Item, such as an offering by a food provider, is requested 110. The Engine determines whether the user data is in a database 112 and whether the Taste Item data is in a database 113. When both the user data and the Taste Item data are found in their respective databases, the matching engine is called 120 and results are rendered 130. If user data is not found in a database, data is obtained from the user 118 and stored in a user data database 620 (FIG. 6). If the Taste Item data is not found in a database, Taste Item data is obtained 116 and stored in a Taste Item data database 520 (FIG. 5).

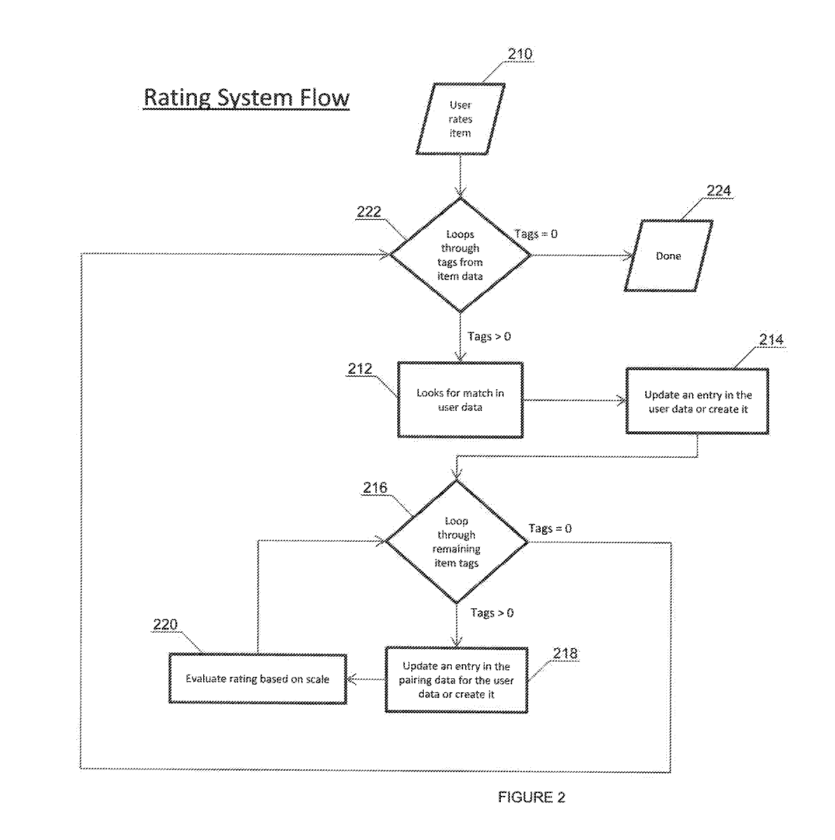

FIG. 2 is a process flowchart for an exemplary embodiment of a rating system of a Recommendation Engine of the present disclosure. A user rates a Taste Item 210. Item data is searched for Tags 222. If no Tag is found the process is done 224. If a first Tag is found, a match is sought in a user preference database 212; the user preference database entry for the Item is updated 214. The engine proceeds to update or record the good pairing or bad pairing of all the remaining Tags against this preference entry appropriately, based on the rating given to the initial Taste Item. The rating is evaluated based on an evaluation scale 220. The process iterates through all the Taste Item Tags until all the combinations between Tags are recorded and the process stops 224.

The rating system of FIG. 2 may be further understood with an example. A user “Pablo” likes tacos. Tacos can be described based on a variety of Item Tags, such as ingredients, textures, cooking methods, taste intensity, etc., some of which Pablo likes and some of which he doesn't. Pablo may prefer some Tags over others and some combinations of Tags over other combinations. The user has a preference database 620 (FIG. 6) that records the frequency and average rating for each Tag the user has expressed any interested in, as well as a pointer to two separate databases “good pairing” and “bad pairing”, to record the combinational relationships between preferences for that user.

If from a rating scale of 1 through 5, Pablo rates a taco made with onion, avocado and pork, a 5, the engine would proceed to first record and/or update the frequency and average rating all these Tags in the user's preference database. Secondly, it would record all the possible combinations as good pairings given a rating threshold of 3, inclusive, and record/update their frequency and average rating. After the iterative process completes, the system would know that user Pablo considers all of the following to be good combinations: taco and onion, taco and avocado, taco and pork, onion and avocado, onion and pork, and avocado and pork.

In another way to illustrate the example, User Pablo has rated 3 tacos that have the following Tags:

Taco 1: [taco, onion, avocado, pork]—Rated a 5

Taco 2: [taco, onion, pork, mango]—Rated a 5

Taco 3: [taco, shrimp, mango]—Rated a 2

In this scenario, the threshold for a good pairing is at 3, inclusive. That user's databases would look like the following:

Preference Database

| | | | | | Good | Bad |
|---|---|---|---|---|---|---|
| Preference | | | | Avg. | Pairing | Pairing |
| ID | User | Tag | Freq | Rating | ID | ID |
| 1 | pablo | taco | 3 | 4 | 1, 2, 3, 4 | 1, 2 |
| 2 | pablo | onion | 2 | 5 | 5, 6, 7, — | |
| 3 | pablo | avocado | 1 | 5 | — | — |
| 4 | pablo | pork | 1 | 5 | — | — |
| 5 | pablo | shrimp | 1 | 2 | — | — |
| 6 | pablo | mango | 2 | 3.5 | — | — |
| — | — | — | — | — | — | — |

Good Pairing Database

| Good | | | |
|---|---|---|---|
| Pairing | Preference | | |
| ID | ID | Tag | Freq |
| 1 | 1 | onion | 2 |
| 2 | 1 | avocado | 1 |
| 3 | 1 | pork | 2 |
| 4 | 1 | mango | 1 |
| 5 | 2 | taco | 2 |
| 6 | 2 | avocado | 1 |
| 7 | 2 | pork | 1 |
| . . . | . . . | . . . | . . . |

Bad Pairing Database

| Bad | | | |
|---|---|---|---|
| Pairing | Preference | | |
| ID | ID | Tag | Freq |
| 1 | 1 | shrimp | 1 |
| 2 | 1 | mango | 1 |
| 3 | 5 | taco | 1 |
| 4 | 5 | mango | 1 |
| 5 | 6 | taco | 1 |
| . . . | . . . | . . . | . . . |

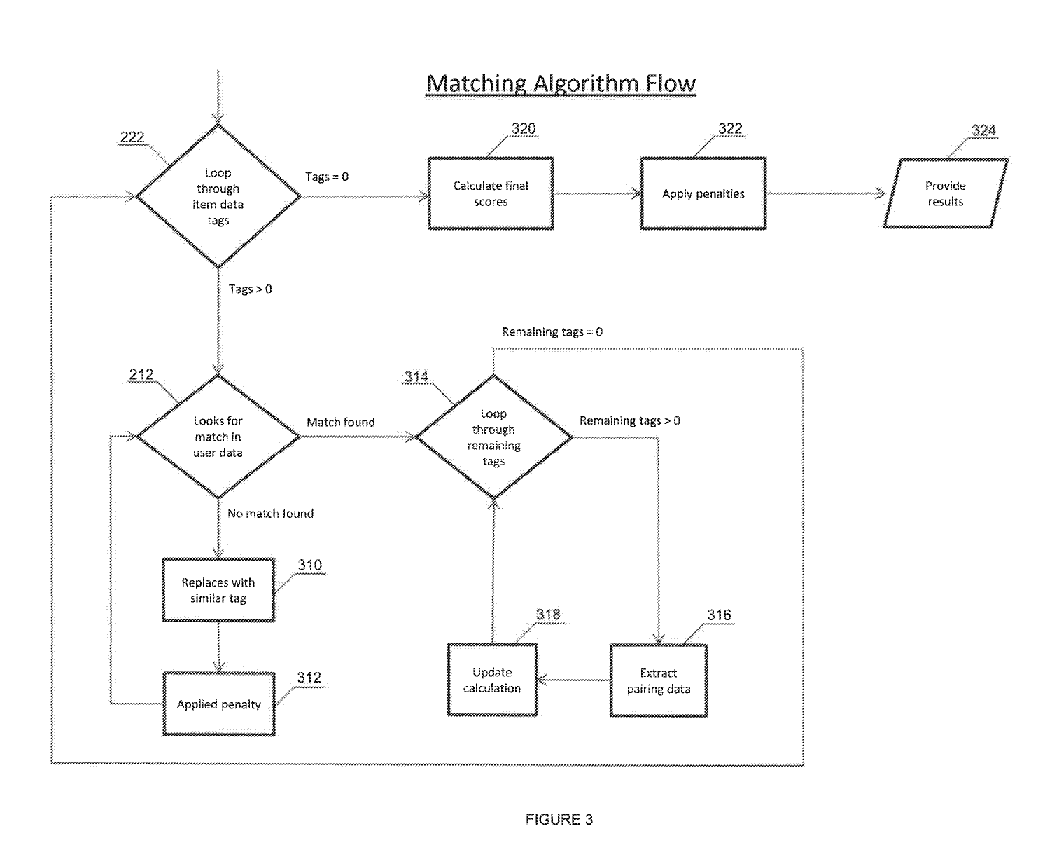

FIG. 3 is a process flow chart for an exemplary embodiment of a matching engine of a Recommendation Engine of the present disclosure. A loop 222 searches for Item Tags in the Item database. If a first Tag is found, the user preference data 620 is searched for a match 212. If no match is found, the Tag is replaced with a similar Tag 310 based on Taste Network data, such as replacing bacon with pork, a penalty is applied 312 to reflect the replacement, and the process steps reiterate. If a match is found 314, the Item data Tag search loop iterates through the remaining Item Tags that were found at 222. If no remaining Tag is found, the process returns to the Item Tag search loop 222 and reiterates for another Item Tag. If a Tag is found, pairing data is extracted 316 and an update calculation is performed 318. After all the combinations have been exhausted (Tags=0), a final score is calculated 320, penalties, if any, are applied, 322 and a result is provided 324.

Another way to describe FIG. 3 is that when a recommendation is calculated, the engine first extracts all the Item Tags 222; it proceeds sequentially to find/match if that Tag is in the user's preference database 212. If there is no match, it replaces it with a similar Tag 310, applies a penalty 312, and tries again until it finds a match in the user's preference database 620, or skips that Tag if nothing is found 320. If a match is found 314 between an Item Tag and an entry in the user's preference database, the entire list of good pairings and bad pairings is loaded 316 and the engine proceeds to scan the remaining Item Tags for a match there. It calculates 318 the weighted frequency of how many good and bad pairings exists in the original set of dish Tags for that specific user.

An example helps further explain FIG. 3. Following the previously defined user databases, here is what would happen if a new ‘taco’ with the following Tags: taco, bacon, avocado would go through the matching engine for user Pablo:

Find 222 if the user has a preference entry for ‘taco’. Since it does 212, it loads the good pairings for ‘taco’ for that user: onion, avocado, pork, mango, as well as the bad pairings: shrimp, mango.

It then scans 314 the rest of the Item Tags, in this case: bacon, avocado, for a good or bad pairing 316. In this case, it finds ‘avocado’ in the good pairings, and updates the calculation 318.

It moves to the next Item Tag 222 and finds if the user has a preference entry for ‘bacon’ 212. Since it doesn't, it replaces it 310 leveraging information on the Taste Network, in this case, with ‘pork’ and loads the good and bad pairings.

It then scans the rest of the Item Tags 222, in this case: avocado, for a good or bad pairing 212, and finds it as a good pairing 314.

These steps are repeated until all the Item Tags are processed for all the possible combinations and a final score is calculated 324. The accumulation of scores over time creates a user preference profile. Additionally, the engine determines or enhances a match between a user and offering based on the taste preferences of multiple users who share other similar traits.

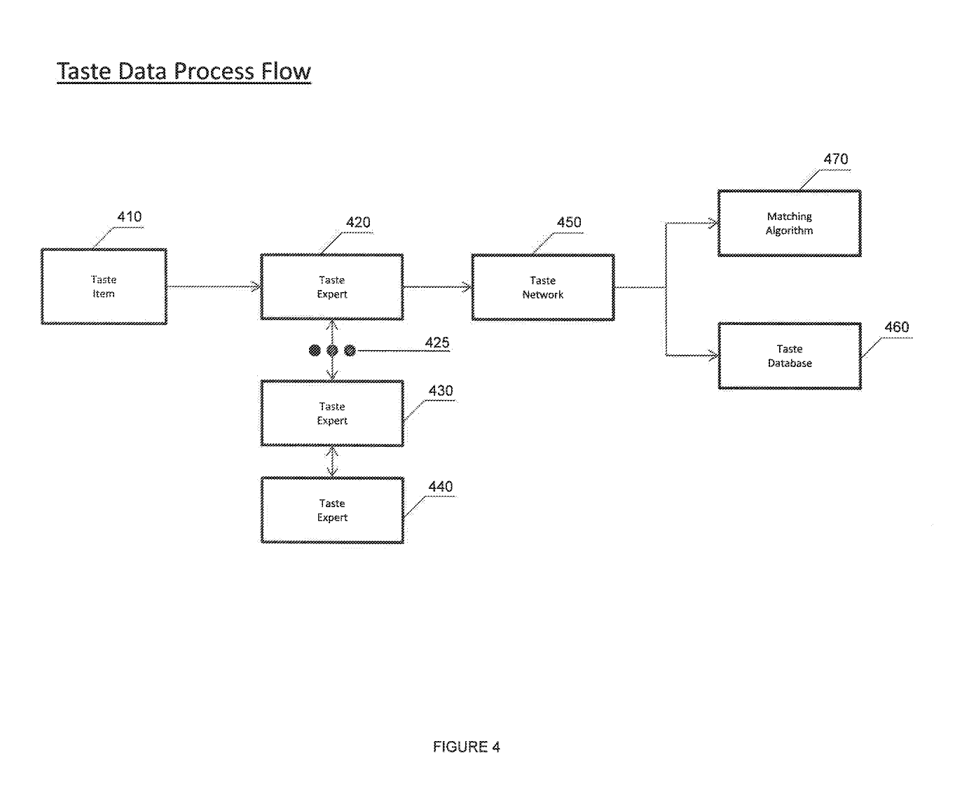

FIG. 4 is a process flowchart for an exemplary embodiment of a taste database of a Recommendation Engine of the present disclosure. A Taste Item is selected 410 and one or more Taste Experts 420, 430, 440 evaluate it and characterize it with Tags from a Taste network 450. Taste network 450 is linked to taste database 460 and matching engine 470. Evaluation by Taste Experts 430, 430, 440, is a dynamic process that may be paused or toggled back and forth, as represented with control icon 425.

FIG. 5 is a diagrammatic illustration of exemplary embodiments of Taste Item databases of a system of the present disclosure. Item data database 510 stores data such as Item name, price, provider name, such as a restaurant, and the like. Database 510 includes or points to taste data, i.e., Tags, database 520.

FIG. 6 is a diagrammatic illustration of exemplary embodiments of user databases of a system of the present disclosure. User data database 610 stores user information such as the user's name, age, and so forth. Database 610 includes or points to user's preference database 620, which stores information, i.e., Tags, about user preferences. Database 620 includes or points to pairing databases 622, 624. Database 622 stores information, i.e., Tags, about good pairings for a user and database 624 stores information, i.e., Tags, about bad pairings for a user.

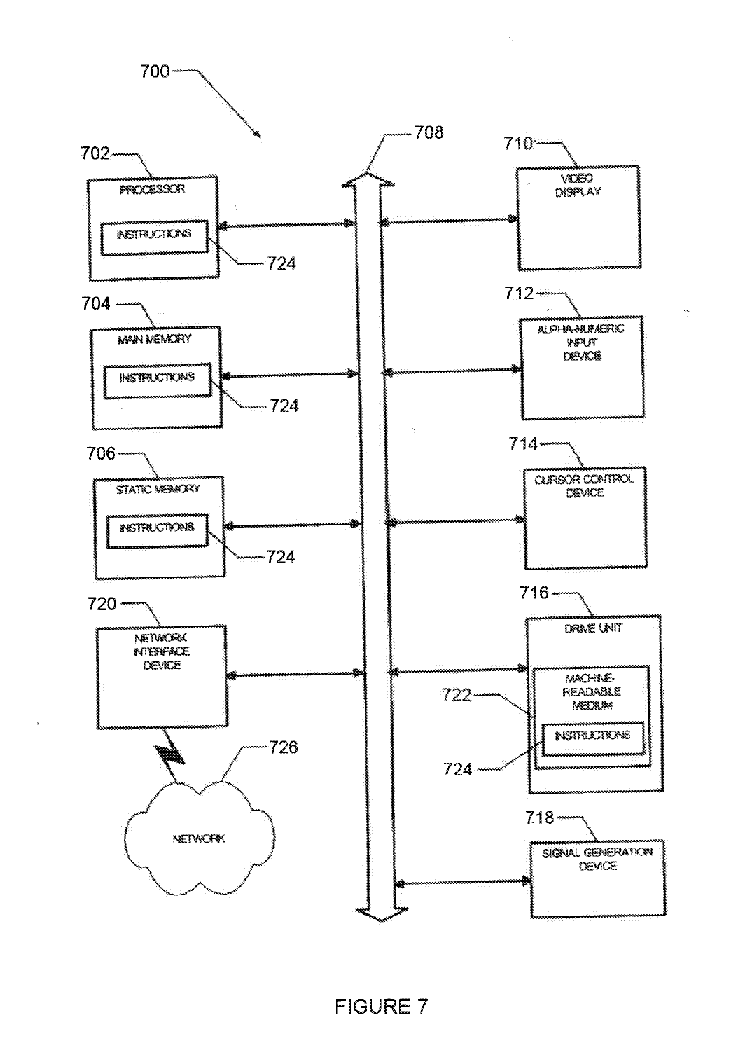

FIG. 7 is a diagrammatic representation of a machine in the form of a computer system 700 within which a set of instructions, when executed, may cause the machine to perform any one or more of the methodologies discussed herein. In some embodiments, the machine operates as a standalone device. In some embodiments, the machine may be connected (e.g., using a network) to other machines. In a networked deployment, the machine may operate in the capacity of a server or a client user machine in server-client user network environment, or as a peer machine in a peer-to-peer (or distributed) network environment. The machine may comprise a server computer, a client user computer, a personal computer (PC), a tablet PC, a set-top box (STB), a Personal Digital Assistant (PDA), a cellular telephone, a smart phone, a mobile device, a wearable Internet device such as a smart watch, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any machine capable of executing a set of instructions (sequential or otherwise) that specify actions to be taken by that machine. It will be understood that a device of the present disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methodologies discussed herein.

The computer system 700 may include a processor 702 (e.g., a central processing unit (CPU), a graphics processing unit (GPU), or both), a main memory 704 and a static memory 706, which communicate with each other via a bus 708. The computer system 700 may further include a video display unit 710 (e.g., a touch screen, a liquid crystal display (LCD), a flat panel, a solid state display, or a cathode ray tube (CRT)). The computer system 700 may include an input device 712 (e.g., a keyboard, touch screen or voice), a cursor control device 714 (e.g., a mouse, touch screen, a remote control), a disk drive unit 716, a signal generation device 718 (e.g., a speaker, Internet connection, or remote control) and a network interface device 720.

The disk drive unit 716 may include a machine-readable medium 722 on which is stored one or more sets of instructions (e.g., software 724) embodying any one or more of the methodologies or functions described herein, including those methods illustrated in herein above. The instructions 724 may also reside, completely or at least partially, within the main memory 704, the static memory 706, and/or within the processor 702 during execution thereof by the computer system 700. The main memory 704 and the processor 702 also may constitute machine-readable media. Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine readable medium containing instructions 724, or that which receives and executes instructions 724 from a propagated signal so that a device connected to a network environment 626 can send or receive voice, video or data, and to communicate over the network 726 using the instructions 724. The instructions 724 may further be transmitted or received over a network 726 via the network interface device 720.

While the machine-readable medium 722 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any non-volatile medium that is capable of storing, encoding or carrying a set of instructions for execution by the machine and that cause the machine to perform any one or more of the methodologies of the present disclosure. The term “machine-readable medium” shall accordingly be taken to include, but not be limited to: solid-state memories such as a memory card or other package that houses one or more read-only (non-volatile) memories, random access memories, or other re-writable (volatile) memories; magneto-optical or optical medium such as a disk or tape; and computer instructions in a non-volatile transmission medium; and/or a digital file attachment to e-mail or other self-contained information archive or set of archives is considered a distribution medium equivalent to a tangible storage medium. Accordingly, the description is considered to include any one or more of a machine-readable medium or a distribution medium, as listed herein and including art-recognized equivalents and successor media, in which the software implementations herein are stored.

Although the present specification describes components and functions implemented in the embodiments with reference to particular standards and protocols, the disclosure is not limited to such standards and protocols. Each of the standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same functions are considered equivalents.

The methods and systems defined in this patent greatly improve the likelihood for an individual to find the best Taste Item for them. Currently, most people select what to eat based on reviews, comments, and ratings from other persons. However this is not only time consuming, but also inefficient as there is still a great chance the individual will not share the same opinion, due to factors like personal preferences, allergies, and dietary restrictions. A personalized taste recommendation system that evolves as it learns directly from the user brings constant value to the consumers as well as the food providers, who can learn exactly what their clients' taste preferences are. This in turn allows them to improve the products and experiences provided to their customers.

Many modifications and other embodiments of the Recommendation Engine described herein will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A method for obtaining food recommendations, the method comprising the steps of:
(a) identifying the food taste preferences of a user using Taste Tags;
(b) storing the user's food taste preferences in a database;
(c) defining a user's good and bad taste pairings from the preferences in the database;
(d) identifying a first food provider that offers one or more Taste Items;
(e) gathering information about the offerings of the first food provider using a Taste Expert;
(f) storing the information about the first food provider's offerings in a database;
(g) comparing the user's taste preferences to the information about the first food provider's offerings by accessing the respective databases;
(h) determining from the comparison what is the match value/score between the first food provider's offerings and the user, based on the user's taste preferences;
(i) identifying a next food provider and repeating steps (d)-(h) for each subsequent food provider.
(j) determining, or enhancing, a match between a user and offering based on the taste preferences of multiple users who share other similar traits.

2. The method of claim 1, further comprising repeating steps (d)-(j) until no food provider is identified.

3. The method of claim 1, wherein the user's taste preferences include the user's dietary requirements.

4. The method of claim 1, further comprising initializing, populating, gathering, evolving, learning, and understanding the user's taste preferences.

5. The method of claim 1, further comprising the step of identifying good pairings and bad pairings for a Taste Item based on the user's taste preferences.

6. The method of claim 1, further comprising the step of evolving a user's taste preferences by analysis of the user's actions, wherein user actions comprise viewing, searching, liking, and rating.

7. The method of claim 1, further comprising the steps of creating and populating a Taste Item database with the curated input of a Taste Expert.

8. The method of claim 1, further comprising the step of producing a recommendation between a user and a Taste Item categorized by the Taste Item's taste.

9. The method of claim 8, further comprising the step of displaying the recommendation to the user in one or more suitable ways.

10. The method of claim 1, wherein the method is executed at least partially by software run on a programmable device having a display screen, the method further comprising the step of displaying on the screen a match between a user and a Taste Item categorized by the Taste Item's taste.

11. The method of claim 1, further comprising the step of categorizing a Taste Item based on Tags, which are part of a predetermined and evolving Taste Network.

12. The method of claim 1, further comprising the step of providing a Taste Expert to create, curate, maintain, and expand a Taste Network.

13. The method of claim 1, wherein the step of gathering information about a food provider's offerings uses methodologies that include but are not limited to one or more interviews with a food provider's Taste Expert.

14. A system for obtaining food recommendations to a user using the user's taste preferences, the system comprising:
(a) at least one taste database that provides a plurality of Taste Tags;
(b) one or more user databases, where in at least one of the one or more user databases further comprising at least one preference database that stores the user's taste preferences toward each Taste Tag in a plurality of preference entries wherein each preference entry comprises a plurality of pairings databases; and
(c) a network to provide communication with the at least one taste database and the one or more user databases.

15. A non-transitory machine readable storage medium having stored there on a computer program for a machine to execute a recommendation engine, the computer program comprising a routine of set instructions for causing the machine to perform the steps of:
(a) identifying the food taste preferences of a user using Taste Tags;
(b) storing the user's food taste preferences in a database;
(c) defining a user's good and bad taste pairings from the preferences in the database;
(d) identifying a first food provider that offers one or more Taste Items;
(e) gathering information about the offerings of the first food provider using a Taste Expert;
(f) storing the information about the first food provider's offerings in a database;
(g) comparing the user's taste preferences to the information about the first food provider's offerings by accessing the respective databases;
(h) determining from the comparison what is the match value/score between the first food provider's offerings and the user, based on the user's taste preferences;
(i) identifying a next food provider and repeating steps (d)-(h) for each subsequent food provider.
(j) determining, or enhancing, a match between a user and offering based on the taste preferences of multiple users who share other similar traits.

16. The non-transitory machine readable storage medium of claim 15, wherein the instructions further cause the machine to further perform the step of repeating steps (d)-(j) until no food provider is identified.

17. The non-transitory machine readable storage medium of claim 15, wherein the instructions further cause the machine to further perform the step of identifying good combinations and bad combinations for a Taste Item based on the user's Taste Network.

18. The non-transitory machine readable storage medium of claim 15, wherein the instructions further cause the machine to further perform the step of evolving a user's taste preferences network by analysis of the user's actions, wherein user actions comprise viewing, searching, liking, and rating.

19. The non-transitory machine readable storage medium of claim 15, wherein the instructions further cause the machine to further perform the step of producing a recommendation between a user and a Taste Item categorized by the Taste Item's taste.

20. The non-transitory machine readable storage medium of claim 15, wherein the instructions further cause the machine to further perform the step of displaying the recommendation to the user in one or more suitable ways.
